(19) United States
(12) Patent Application Publication
(10) Pub. No.: US 2021/0000703 A1
(43) Pub. Date: Jan. 7, 2021

(54) MULTIPLE DUROMETER COSMETIC PAD

(71) Applicant: HCT GROUP HOLDINGS LIMITED, SHEUNG WAN (CN)

(72) Inventor: DENIS PIERRE MAURIN, LOS ANGELES, CA (US)

(73) Assignee: HCT GROUP HOLDINGS LIMITED, SHEUNG WAN (CN)

(21) Appl. No.: 16/918,389

(22) Filed: Jul. 1, 2020

Related U.S. Application Data
(60) Provisional application No. 62/870,081, filed on Jul. 3, 2019.

(51) Int. Cl. A61K 8/02; A45D 34/04; A45D 33/34; A61Q 19/10

(52) U.S. Cl. CPC A61K 8/0208; A45D 34/04; A61K 2800/87; A61Q 19/10; A45D 2200/1018; A45D 33/34

(57) ABSTRACT

A cosmetic pad, which may be useful, for example, for cleansing or other cosmetic-related activity, includes an upper portion, a lower portion coupled to the upper portion, and a non-porous covering. The non-porous covering is disposed around at least the upper portion. The upper portion has a lower durometer than the lower portion.

### CROSS REFERENCE TO RELATED APPLICATIONS

The present application claims the benefit of and priority to U.S. Provisional Patent Application Ser. No. 62/870,081, filed on Jul. 3, 2019, titled MULTIPLE DUROMETER COSMETIC PAD, the disclosure of which is incorporated herein by reference.

### TECHNOLOGY FIELD

The present application relates generally to a cosmetic pad, and in particular, to a soft pad for cleansing or applying a cosmetic product.

### BACKGROUND

There are numerous cleansing pads and applicators for liquid, cream, and powder cosmetic products available, such as sponges and puffs. Such sponges and puffs are often porous, resulting in absorption of the product and skin oils. The absorption of product into the sponge results in loss of product, and the absorption of skin oils may lead to bacterial growth, requiring thorough cleaning. Cleaning the porous sponge may be problematic due to the amount of product and skin oils embedded within the pores of the sponge. Although different pads and applicators exist, there is a continuing need for more and different cosmetics pads and applicators.

### SUMMARY

According to one example of the present disclosure, a cosmetic pad comprises an upper portion, a lower portion coupled to the upper portion, and a non-porous covering disposed around at least the upper portion, wherein the upper portion has a lower durometer than the lower portion.

Alternatively, or additionally, in another example, the upper portion has a shore hardness between 20 and 40 on the OOO shore scale, and the lower portion has a shore hardness between 45 and 85 on the OOO shore scale.

Alternatively, or additionally, in another example, the upper portion has a shore hardness between 25 and 35 on the OOO shore scale, and the lower portion has a shore hardness between 55 and 75 on the OOO shore scale.

Alternatively, or additionally, in another example, the upper portion has a shore hardness of 30 on the OOO scale and the lower portion has a shore hardness of 65 on the OOO scale.

Alternatively, or additionally, in another example, the upper portion and the lower portion are made of different materials.

Alternatively, or additionally, in another example, the upper portion and the lower portion are made of the same material but with a differing shore hardness.

Alternatively, or additionally, in another example, at least one of the upper portion and the lower portion is made of silicone, thermoplastic elastomers, rubber, or nitrile butadiene rubber.

Alternatively, or additionally, in another example, the upper portion is made of silicone and the lower portion is not made of silicone.

Alternatively, or additionally, in another example, the covering has a thickness of between 0.005 mm and 0.1 mm.

Alternatively, or additionally, in another example, the covering includes polyurethane.

Alternatively, or additionally, in another example, an entirety of the cosmetic pad is latex-free.

Alternatively, or additionally, in another example, the upper portion is cylindrical with a domed top surface, and the lower portion is cylindrical.

Alternatively, or additionally, in another example, the cosmetic pad further comprises a plurality of protrusions on at least a top surface of the upper portion.

Alternatively, or additionally, in another example, the plurality of protrusions are formed by both the upper portion and the covering.

Alternatively, or additionally, in another example, the plurality of protrusions are formed by only the covering.

According to another example, a cosmetic pad comprises an upper portion defining a plurality of protrusions, a lower portion coupled to the upper portion, and a non-porous covering disposed around at least the upper portion, wherein the upper portion has a lower durometer than the lower portion.

Alternatively, or additionally, in another example, the upper portion has a shore hardness between 25 and 35 on the OOO shore scale, and the lower portion has a shore hardness between 55 and 75 on the OOO shore scale.

Alternatively, or additionally, in another example, the covering has a thickness of between 0.005 mm and 0.1 mm.

Alternatively, or additionally, in another example, at least one of the upper portion and the lower portion is made of silicone, thermoplastic elastomers, rubber, or nitrile butadiene rubber, and the covering includes polyurethane.

According to another example, a cosmetic pad comprises an upper portion defining a plurality of protrusions, a lower portion coupled to the upper portion, and a non-porous covering disposed around at least the upper portion, wherein the upper portion has a shore hardness between 20 and 40 on the OOO shore scale, and the lower portion has a shore hardness between 45 and 85 on the OOO shore scale, wherein the non-porous covering has a thickness of between 0.005 mm and 0.1 mm.

The above summary of some example embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 is a perspective view of an illustrative cosmetic pad;

FIG. 2 is a top view of the cosmetic pad of FIG. 1;

FIG. 3 is a bottom view of the cosmetic pad of FIG. 1;

FIG. 4 is a side view of the cosmetic pad of FIG. 1; and

FIG. 5 is a cross-sectional view taken along line 5-5 in FIG. 4.

### DETAILED DESCRIPTION

Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure. The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise. As used in this specification and the appended claims, the terms “approximately” and “substantially” may indicate a range of values within +/−5% of a stated value or position.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

For convenience, the present disclosure may be described using relative terms including, for example, left, right, top, bottom, front, back, upper, lower, up, and down, as well as others. It is to be understood that these terms are merely used for illustrative purposes and are not meant to be limiting in any manner.

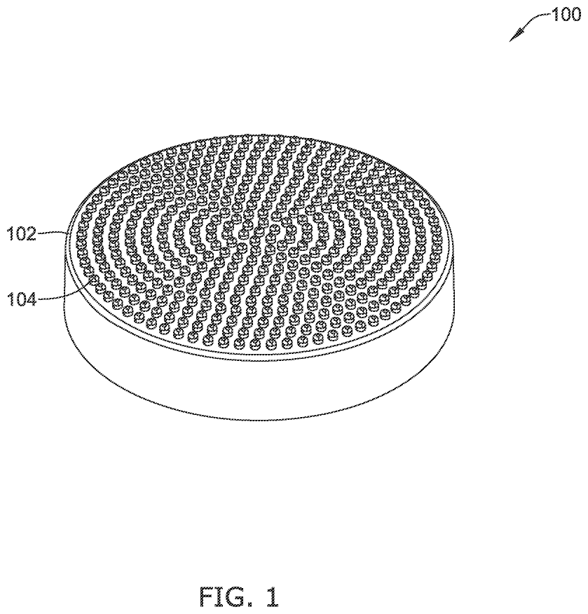

FIG. 1 is a perspective view of a cosmetic pad 100 according to embodiments disclosed herein. The cosmetic pad 100 may have a top surface 102 with a surface texture including a plurality of bumps, bristles, or protrusions 104. The overall shape of the cosmetic pad 100 may be cylindrical with a domed top, as shown in the figures, however it will be understood that the cosmetic pad 100 may have any shape, such as, but not limited to egg-shaped, spherical, rectangular, triangular, capsule-shaped, cylindrical with a flat top, etc.

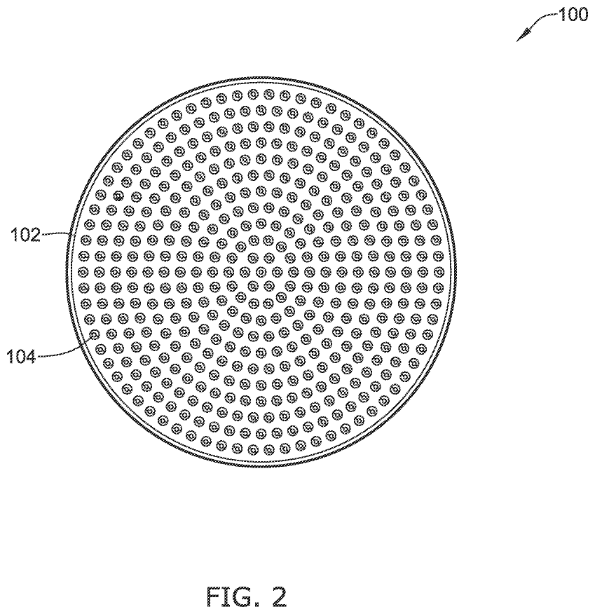

The plurality of protrusions 104 may be arranged over the entire top surface 102, as shown in FIG. 2. In other embodiments, the protrusions 104 may be disposed over only a portion of the top surface 102, for example, in the middle portion only or over only one half of the circular top surface 102. In embodiments in which the protrusions 104 cover only a part of the top surface 102, the remainder of the top surface 102 may have a surface texture different from the protrusions 104, such as a roughened surface, ridges, cross-hatching, a sanded surface, etc. In other embodiments, the remainder of the top surface 102 may be devoid of any surface texture, providing a smooth surface.

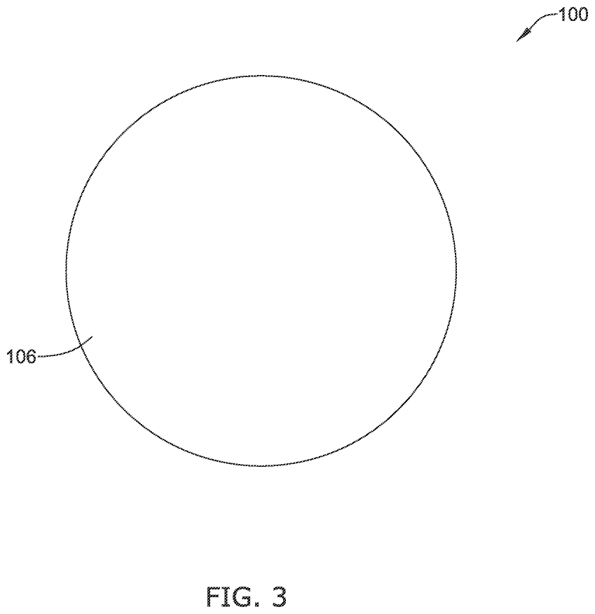

The cosmetic pad 100 may have a bottom surface 106 that is substantially flat. In some embodiments, the bottom surface 106 may be devoid of surface texture, as shown in FIG. 3. In other embodiments, the bottom surface 106 may have a surface texture such as a roughened surface, ridges, cross-hatching, a sanded surface, etc.

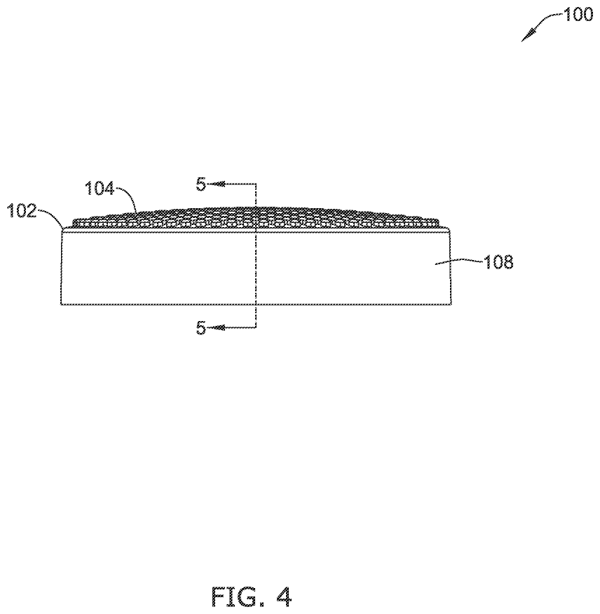

FIG. 4 is a side view of the cosmetic pad 100, showing the protrusions 104 on the top surface 102. The side surface 108 may be devoid of any surface texture, as shown in FIG. 4. In other examples, at least part of the side surface 108, such as the side surface of the upper portion 110, may include a plurality of protrusions 104 (not shown). In still further examples, the side surface 108 may have a surface texture such as a roughened surface, ridges, cross-hatching, a sanded surface, etc.

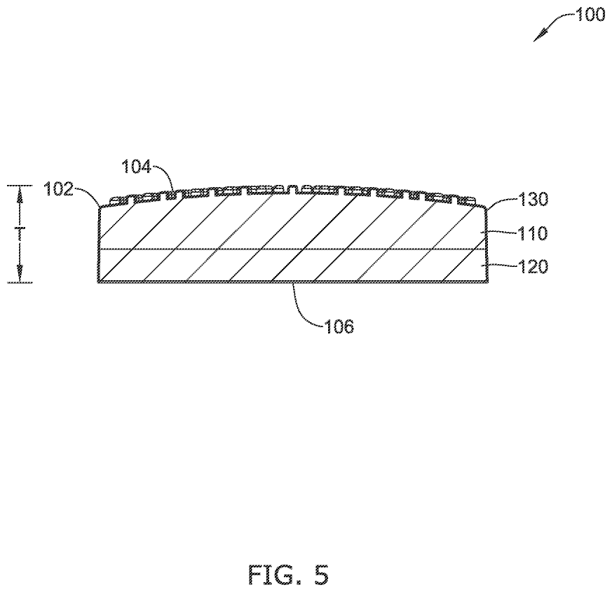

The cosmetic pad 100 may include an upper portion 110, a lower portion 120, and a covering 130, as shown in the cross-section of FIG. 5. The covering 130 may surround the entire cosmetic pad 100 and may define the top surface 102, the side surface 108, and the bottom surface 106. In other examples, the covering 130 may be disposed only on the upper portion 110, defining the top surface 102 and at least a portion of the side surface 108. In still further examples, the covering 130 may define the top surface 102, the entire side surface 108, but not the bottom surface 106. The cosmetic pad 100 may have a diameter of between 10 mm and 100 mm and a thickness T measured between the bottom surface 106 and the highest point of the tallest projection 104 of between 5 mm and 30 mm. In further examples, the cosmetic pad 100 may have a diameter of around 40 mm with a thickness T of around 8 mm.

The upper portion 110 may be made of a softer material than the lower portion 120. The upper portion 110 with the protrusions 104 may be soft for applying product to the face or for cleansing. In order to prevent the soft upper portion 110 from coming apart at the bottom where the user grasps it or where it attaches to a holder, the lower portion 120 may be made of a harder material to prevent deformation and stretching that might tear the covering 130.

In some examples, the protrusions 104 may be formed by both the upper portion 110 and the covering 130, as illustrated in FIG. 5. In other examples, the protrusions 104 may be formed by only the covering 130.

The hardness of the upper portion 110 and the lower portion 120 are different. In some examples, the upper portion 110 may be made of a material having a hardness of between 20 and 40 on the shore OOO scale, and the lower portion 120 may be made of a material having a hardness of between 45 and 85 on the shore OOO scale. In other examples, the upper portion 110 may be made of a material having a hardness of between 25 and 35 on the shore OOO scale, and the lower portion 120 may be made of a material having a hardness of between 55 and 75 on the shore OOO scale. In a further example, the upper portion 110 may be made of a material having a hardness of around 30 on the shore OOO scale, and the lower portion 120 may be made of a material having a hardness of around 65 on the shore OOO scale.

The upper portion 110 and the lower portion 120 may be made of the same material with differing hardness measurements, or they may be made from different materials. In some examples, the upper portion 110 and/or the lower portion 120 may be made of silicone, thermoplastic elastomers (TPE), rubber, or nitrile butadiene rubber (NBR).

The covering 130 may be made of a non-porous, closed-cell material, preventing any liquid from coming into contact with the upper portion 110 and lower portion 120. In some examples, the covering 130 may be a thin polyurethane (PU) layer. In some examples, the covering 130 may have a thickness of between 0.005 mm to 0.1 mm. In other examples, the covering 130 may have a thickness of between 0.03 mm to 0.05 mm. In one example, the covering 130 may have a thickness of 0.04 mm. The entire cosmetic pad 100, including the upper portion 110, the lower portion 120, and the covering 130, may be latex-free.

The upper portion 110 and lower portion 120 may be attached during the manufacturing process. In some examples, the two portions may be molded. The mold may be positioned upside down with the covering 130 disposed within it, then a first material, which will form the upper portion 110, is poured or injected into the mold and cooled. After cooling, a second material, which will form the lower portion 120, is poured or injected onto the first material. If the second material is a gel then the hot gel is poured onto the cooled first material, thus bonding the second material to the first material. If the second material is injected then the injection bonds because it is molten during injection. In other examples, the upper portion 110 and the lower portion 120 may be formed separately and attached to each other via hot-melt adhesive, thermo-compression bonding, or any other joining process that achieves a stable and secure attachment. The covering 130 may then be attached via molding, dipping, spraying, etc.

In some examples, the cosmetic pad 100 may be held directly by the user, generally by gripping the lower portion 120. In other examples, the cosmetic pad may be disposed in a holder (not shown). The holder may have a cup shape covering the bottom surface 106 and at least part of the side surface 108. In some examples, the holder may include a handle that is either held by the user or that is configured to receive one or more fingers of the user.

The cosmetic pad 100 may be used with a cleanser to clean and/or exfoliate the skin, especially on the face. The protrusions 104 provide a scrubbing and exfoliating surface. The non-porous surface of the cosmetic pad 100 allows for substantially all of the cleanser product to be released, avoiding loss of product into the cosmetic pad 100. This is an advantage over porous pads such as sponge, cloth, or flocked pads that may absorb relatively large amounts of product in their pores and between fibers which may result in staining of the pad and retention of product within the pad, which can reduce the ability of the pad to effectively apply cleanser product, harbor bacteria, contaminate freshly applied product with old (possibly degraded) product, or simply require a new pad for each use.

The cosmetic pad 100 may be intended to be reusable and washable. The top surface 102 with its plurality of protrusions 104 may provide a textured surface for cleansing of the skin. The non-porous surface of the cosmetic pad 100 provides a surface for cleansing the skin without absorbing product into the cosmetic pad 100. This may aid in cleansing with a small amount of product without loss of any product into the cosmetic pad 100.

The cosmetic pad 100 may also be used to apply cosmetic products such as concealer, foundation, or other liquid, cream, or powder products. For a cosmetic product applicator, the cosmetic pad 100 may have a substantially smooth top surface 102, either devoid of any texture, or with a minimal texture, such as low ridges or cross-hatching. The cosmetic pad 100 may be used to pick up cosmetic material on its surface and then release the cosmetic material onto the skin. The cosmetic pad 100 is able to release substantially all of the cosmetic material that has been picked up due to the non-porous nature of the top surface 102.

The ability of the cosmetic pad 100 to release substantially all of the cosmetic material from the surface during use makes the cosmetic pad 100 easy to clean. The surface may be cleaned by wiping with a tissue or cloth, or washing with a mild cleanser. This results in the cosmetic pad 100 surface remaining clean while in storage, thus preventing growth of bacteria on the surface, or contamination of fresh product with old product remaining on the surface, or accumulation of substances that may stain the cosmetic pad 100. The ability of the cosmetic pad 100 to release substantially all of the cosmetic material from the surface during use also prevents waste of product. As no product is absorbed by the cosmetic pad 100, the cosmetic pad is advantageously used with high value products without waste.

In some examples, the upper portion 110 may be a polymer composition such as a gel which exhibits both liquid and solid characteristics, depending upon the particular composition; that is, the gel can range from a semi-liquid to a semi-solid. In some aspects, a gel composition may contain a three-dimensional network of cross-linked molecular chains (gels), or it may merely behave as if it contained such a network (gelloids). The coating 130 may be a polymer or other material such as polyurethane capable of containing even semi-liquid gels, and providing resilient structure for the gel which permits a desired amount of deformation, while enough resiliency to return to its original shape. The upper portion 110 may also include phase change materials which are useful in conveying the cooling sensation, since phase change materials, by definition absorb heat energy when changing phases. The gel of embodiments described in this document may be any liquid-extended polymer or a gelatinous composition having a molecular weight and hardness characteristics that allow the gel to deform yet have a resilience that allows it to rebound into its original shape quickly, such as within one second or less, when the load is removed. Examples of gels used in aspects of the invention include, without limitation, oil-extended triblock copolymer compositions such as that disclosed in U.S. Pat. Nos. 3,485,787, 3,676,387, 3,827,999, 4,176,240, 4,259,540, 4,351,913, 4,432,607, 4,492,428, 4,497,538, 4,509,821, 4,709,982, 4,716,183, 4,833,193, 4,942,270, 5,149,736, 5,331,036, and 5,994,450; and thermoplastic elastomer gelatinous compositions such as that disclosed in U.S. Pat. Nos. 4,369,284, 4,618,213, 5,262,468, 5,508,334, 5,153,254, 5,334,646, 5,239,273, 5,475,890, and 5,336,708. Each of the above references is hereby incorporated by reference.

The gel can comprise any gel that is stable, nontoxic, and generally known to provide a cushioning effect while maintaining a degree of structural stability and support. Polyurethane gels are particularly useful. Further, other gels that are resistant to hardening over time, have limited expandability, and are resistant to substance degradation (e.g., from migration of volatile agents, such as plasticizers) could also be useful as the gel in the present invention.

Examples of polyurethane gels capable of use according to the invention are disclosed in U.S. Pat. No. 6,191,216, United States Published Patent Application No. 2004/0058163 (application Ser. No. 10/618,558) and U.S. Published Patent Application No. 2004/0102573 (application Ser. No. 10/656,778), which are incorporated herein by reference.

Examples of other types of gels useful according to the invention are disclosed in U.S. Pat. Nos. 4,404,296, 4,456,642, and U.S. Published Patent Application No. 2007/0061978 (application Ser. No. 11/365,473), which are incorporated herein by reference.

Although the invention has been described with reference to exemplary embodiments, it is not limited thereto. Those skilled in the art will appreciate that numerous changes and modifications may be made to the embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A cosmetic pad comprising:
an upper portion;
a lower portion coupled to the upper portion; and
a non-porous covering disposed around at least the upper portion;
wherein the upper portion has a lower durometer than the lower portion.

2. The cosmetic pad of claim 1, wherein the upper portion has a shore hardness between 20 and 40 on the OOO shore scale, and the lower portion has a shore hardness between 45 and 85 on the OOO shore scale.

3. The cosmetic pad of claim 1, wherein the upper portion has a shore hardness between 25 and 35 on the OOO shore scale, and the lower portion has a shore hardness between 55 and 75 on the OOO shore scale.

4. The cosmetic pad of claim 3, wherein the upper portion has a shore hardness of 30 on the OOO scale and the lower portion has a shore hardness of 65 on the OOO scale.

5. The cosmetic pad of claim 1, wherein the upper portion and the lower portion are made of different materials.

6. The cosmetic pad of claim 1, wherein the upper portion and the lower portion are made of the same material but with a differing shore hardness.

7. The cosmetic pad of claim 1, wherein at least one of the upper portion and the lower portion is made of silicone, thermoplastic elastomers, rubber, or nitrile butadiene rubber.

8. The cosmetic pad of claim 1, wherein the upper portion is made of silicone and the lower portion is not made of silicone.

9. The cosmetic pad of claim 1, wherein the covering has a thickness of between 0.005 mm and 0.1 mm.

10. The cosmetic pad of claim 1, wherein the covering includes polyurethane.

11. The cosmetic pad of claim 1, wherein an entirety of the cosmetic pad is latex-free.

12. The cosmetic pad of claim 1, wherein the upper portion is cylindrical with a domed top surface, and the lower portion is cylindrical.

13. The cosmetic pad of claim 1, further comprising a plurality of protrusions on at least a top surface of the upper portion.

14. The cosmetic pad of claim 13, wherein the plurality of protrusions are formed by both the upper portion and the covering.

15. The cosmetic pad of claim 13, wherein the plurality of protrusions are formed by only the covering.

16. A cosmetic pad comprising:
an upper portion defining a plurality of protrusions;
a lower portion coupled to the upper portion; and
a non-porous covering disposed around at least the upper portion;
wherein the upper portion has a lower durometer than the lower portion.

17. The cosmetic pad of claim 16, wherein the upper portion has a shore hardness between 25 and 35 on the OOO shore scale, and the lower portion has a shore hardness between 55 and 75 on the OOO shore scale.

18. The cosmetic pad of claim 16, wherein the covering has a thickness of between 0.005 mm and 0.1 mm.

19. The cosmetic pad of claim 16, wherein at least one of the upper portion and the lower portion is made of silicone, thermoplastic elastomers, rubber, or nitrile butadiene rubber, and the covering includes polyurethane.

20. A cosmetic pad comprising:
an upper portion defining a plurality of protrusions;
a lower portion coupled to the upper portion; and
a non-porous covering disposed around at least the upper portion;
wherein the upper portion has a shore hardness between 20 and 40 on the OOO shore scale, and the lower portion has a shore hardness between 45 and 85 on the OOO shore scale, wherein the non-porous covering has a thickness of between 0.005 mm and 0.1 mm.
